(19) United States
(12) Patent Application Publication
(10) Pub. No.: US 2006/0199203 A1
(43) Pub. Date: Sep. 7, 2006

(54) Extraction of high-purity DNA and RNA

(75) Inventor: Richard A. Hurt, Oak Ridge, TN (US)

Correspondence Address: BIRCH STEWART KOLASCH & BIRCH, PO BOX 747, FALLS CHURCH, VA, 22040-0747, US

(73) Assignee: ATOM SCIENCES, Oak Ridge, TN (US)

(21) Appl. No.: 11/356,152

(22) Filed: Feb. 17, 2006

Related U.S. Application Data
(60) Provisional application No. 60/654,081, filed on Feb. 18, 2005.

(51) Int. Cl. C12Q 1/68; C12N 1/08

(52) U.S. Cl. 435/6; 435/270

(57) ABSTRACT

The present invention is directed to a method of extracting nucleic acids from samples containing prokaryotic cells and/or eukaryotic cells by (a) lysing the cells under conditions that minimize RNA expression or enzymatic degradation of RNA; (b) dispersing and diluting the lysed cells; (c) mixing the dispersed and diluted lysed cells with a buffered solution containing cationic and anionic detergents; (d) separating non-nucleic acid contaminants from the nucleic acids by mixing with an organic extraction solvent to generate an emulsion that separates into an organic phase, an interface containing the contaminants and an aqueous phase containing the nucleic acids; (e) separating the phases, and retaining the aqueous phase; and (f) precipitating the nucleic acids in the aqueous phase.

### FIELD OF THE INVENTION

This invention relates to the fields of nucleic acids extraction and purification.

### BACKGROUND OF THE INVENTION

RNA extraction and purification from samples containing high levels of organic matter present special problems. Organic contaminants such as humic and fulvic acids, which are heterogeneous mixtures of polymerized organic matter created by chemical and biological degradation of biological residues, co-fractionate with RNA when standard pH-based differential organic extraction techniques are used to separate RNA from DNA [Chomczynski and Sacchi, Anal. Biochem., 162:156 (1987)] Soil is particularly problematic in this respect because soil organic matter contains a large quantity of carboxylic acid groups and the resulting polyanionic characteristics of soil organic matter are very similar to the characteristics caused by the anionic phosphate groups in the backbone of nucleic acids. If these organic contaminants are not removed from nucleic acid samples, they inhibit enzymatic manipulation [Lovell and Piceno, J. Microbiol. Methods, 20:161, (1994); Malik et al., J. Microbiol. Meth., 20:183, (1994); Purdy et al., Appl. Environ. Microbiol., 62:3905, (1996); Selenska and Klingmüller, Microb Releases, 1:41, (1992); Zhou et al., Appl. Environ. Microbiol., 62:316, (1996)], reduce hybridization efficiency [Alm et al., Appl. Environ. Microbiol., 66:4547, (2000); Moran, et al., Appl. Environ. Microbiol., 59:915, (1993)], cause error in nucleic acids quantification [Dell'Anno et al., Appl. Environ. Microbiol., 64:3238, (1998); Lovell and Piceno, J. Microbiol. Methods 20:161-174, (1994); Moran, et al., Appl. Environ. Microbiol., 59:915, (1993); Ogram et al., Appl. Environ. Microbiol., 61:763, (1995); Purdy et al., Appl. Environ. Microbiol., 62:3905, (1996)], and damage the nucleic acids. While several DNA purification approaches and commercial extraction kits have proven effective for removing organic material from samples, none of these approaches can produce RNA that is amenable to enzymatic manipulation from samples with significant fulvic acid or humic acid content.

A number of techniques have been used to reduce the problems caused by organic contaminants in RNA extractions. Repetitive organic extraction removes humin but does little to remove humic acids-like organic contaminants. Repeated extraction also depletes the quantity of recovered nucleic acids. Separation of RNA from DNA using a CsCl gradient technique results in a large quantity of organic contaminants in the resulting RNA pellet [Selenska and Klingmüller, Microb Releases, 1:41, (1992)]. Precipitation of nucleic acids using polyethylene glycol and cetyltrimethyl ammonium bromide (CTAB) [Coolen and Overmann, Appl. Environ. Microbiol., 64:4513, (1998); Ogram et al., J. Microbiol. Methods, 7:57, (1987); Selenska and Klingmüller, Microb Releases, 1:41, (1992)], precipitation of organic contaminants with ammonium acetate or potassium acetate [Lee et al., Appl. Environ. Microbiol., 62:3787, (1996); Ogram et al., J. Microbiol. Methods, 7:57, (1987)], adsorption of nucleic acids to polyvinylpyrrolidone [Coolen and Overmann, Appl. Environ. Microbiol., 64:4513, (1998)], and molecular exclusion resin chromatography [Duarte et al., J. Microbiological Methods, 32:21, (1998); Moran, et al., Appl. Environ. Microbiol., 59:915, (1993)] have all been shown to reduce co-precipitation of contaminating organic matter. However, these approaches do not provide complete purification and are typically followed by additional methods for further purification. Where quantities recovered before and after purification have been reported, the vast majority of soil RNA is lost during the purification process [Ogram et al., Appl. Environ. Microbiol., 61:763, (1995)].

Soil extraction procedures based on heating slurries yield nucleic acid preparations contaminated with discoloring organic matter [Hayes, M. H. B. In Humic Substances in Soil, Sediment, and Water (G. R. Aiken, D. M. McKnight, R. L. Wershaw and P. MacCarthy, eds.) John Wiley & Sons NY, (1989)]. These methods are used because the greater DNA yields imply reduced sampling bias of microbial communities, and high molecular weight soil DNA can be recovered (Hurt et al., Appl. Environ. Microbiol., 67:4495, (2001); Leff et al., Appl. Environ. Microbiol., 61:1141, (1995); Rondin et al., Appl. Environ. Microbiol., 66:2541, (2000); Zhou et al., Appl. Environ. Microbiol., 62:316, (1996)]. Rapid bead milling procedures co-extract minimal organic material with RNA preparations [Borneman and Triplett, Soil Biol. Biochem., 29:1621, (1997)]. However, bead milling is not suitable for recovery of high molecular weight DNA and the method requires placing the sample in solution prior to lysis, thus allowing a change in expressed mRNA levels. A method for simultaneous recovery of total RNA and high molecular weight DNA was recently reported by the inventor and coworkers [Hurt et al., Appl. Environ. Microbiol., 67:4495, (2001)]. In this work, A-horizon soils from eastern Tennessee required extraordinary effort to prepare mRNA of sufficient purity for enzymatic manipulation. These efforts included passage through Sephadex G-75 molecular exclusion resin [Moran, et al., Appl. Environ. Microbiol., 59:915, (1993)] followed by a second organic extraction prior to nucleic acid fractionation using QIAGEN anion exchange resin. Following fractionation, RNA samples were re-purified using QIAGEN anion exchange resin.

Electrophoresis is the only approach that has been proven to completely remove organic contaminants from soil nucleic acid preparations [Ausubel et al., (eds.), Current Protocols in Molecular Biology, John Wiley & Sons, Inc., New York, N.Y. (1995); Malik et al., J. Microbiol. Meth., 20:183, (1994); Zhou et al., Appl. Environ. Microbiol., 62:316, (1996)]. However, the heterogeneous size distribution of RNA molecules precludes routine use of electrophoretic separation for purification. Exhaustive dialysis has been successfully applied to the purification of RNA, but this approach is tedious [Ogram et al., Appl. Environ. Microbiol., 61:763, (1995)]. Binding nucleic acids to anion exchange resins followed by washing with a solution that does not remove the nucleic acids and then eluting the purified nucleic acids is a common method for nucleic acid purification. The inventor has tested several anion exchange resins for RNA purification using the standard nucleic acid binding, washing, and elution sequence of steps currently applied in the art, and found that residual organic matter co-eluted with the RNA in all cases.

Conventional methods for nucleic acid purification use quaternary amine anion exchange resins to bind nucleic acids and then elute the sample. However, the inventor has shown that organic contaminants also bind to these exchange resins over a broad range of pH and ionic strength. The results also showed that the majority of this bound organic material elutes under conditions suitable for the elution of RNA from the anion exchangers, making purification extremely difficult. Significantly, the majority of the organic material binds to the anion exchange resins even under conditions where nucleic acids, including RNA, do not bind.

The present invention uses an inverted strategy wherein the anion exchange resin is modified with acetate ions or chloride ions, which inhibit binding of nucleic acids but allow negatively charged groups on soil organic matter that are known to soil chemists to be primarily carboxylate groups to bind competitively to the resin. Tests have shown that the competitive binding strategy improved the purity of nucleic acids in a wide variety of samples. In some cases, additional organic material could be removed from the nucleic acid sample with conventional molecular exclusion resins as a supplement to the competitive binding technique. Simultaneous application of both the newly developed anion exchange purification procedure and the conventional molecular exclusion purification method can be performed in an optimized resin mixture. Tests have shown that this simultaneous approach gave better results than application of the two methods separately. The mechanism for this improvement is not known. A potential explanation is that when organic material is exposed to the competitive binding resin (quaternary amine modified with acetate, chloride, or other negatively charged counter-ion), it fluctuates between attachment to the nucleic acid, attachment to the anion exchange resin, and an unbound state. In the presence of the molecular exclusion resin, smaller organic molecules that exist in this free state are trapped in the molecular exclusion resin yielding improved nucleic acid purification.

### SUMMARY OF THE INVENTION

The present invention is directed to a method of extracting nucleic acids from samples containing prokaryotic cells and/or eukaryotic cells by:

(a) lysing the cells in the sample under conditions that minimize RNA expression or enzymatic degradation of RNA;

(b) dispersing and diluting the lysed cells;

(c) mixing the dispersed and diluted lysed cells with a buffered solution containing cationic and anionic detergents, wherein the anionic detergent facilitates binding of carboxylate containing materials and ionic binding of the nucleic acids is inhibited by the cationic detergent;

(d) separating non-nucleic acid contaminants from the nucleic acids by mixing with an organic extraction solvent to generate an emulsion that separates into an organic phase, an interface containing the contaminants and an aqueous phase containing the nucleic acids;

(e) separating the phases, and retaining the aqueous phase; and

(f) precipitating the nucleic acids in the aqueous phase followed by dissolving the precipitate in nuclease-free solvent.

In an alternative embodiment, the present invention is directed to a method for simultaneously extracting RNA and DNA from samples containing prokaryotic cells and/or eukaryotic cells by:

(a) lysing sample cells;

(b) diluting the lysed cells;

(c) separating cell proteins and contaminant organic matter from the cell nucleic acids by mixing with an organic extraction solvent to generate an emulsion that separates into an organic phase, an interface containing the contaminants and an aqueous phase containing the nucleic acids,

(d) separating the phases formed in step (c) with the aqueous phase being retained;

(e) precipitating the nucleic acids from the aqueous phase followed by dissolving the nucleic acids in a nuclease-free solvent; and

(f) optionally further purifying the nucleic acids by passing the dissolved nucleic acids through a mixture of molecular exclusion resin and anion exchange resin, wherein any remaining organic contaminants are captured while the nucleic acids pass through.

### BRIEF DESCRIPTION OF THE DRAWINGS

FIG. 1 shows electrophoretic profiles of to nucleic acids unpurified and purified using the method of the invention from two deciduous forest A-horizon soil samples (1-2), two flood plain sediment samples (3-4), and two grassy rhizosphere soil samples (5-6). The component removed by the purification process of the invention contains fragmented DNA, suggesting that it is likely extracellular DNA at some stage of the humification process, and which is a potent PCR inhibitor.

FIG. 2 shows the electrophoresis gel image of glnA RTPCR products generated from deciduous forest A-Horizon soil samples using the purification method of the invention. RNA was separated from the DNA, quantified, and a two-fold dilution series was prepared. Reverse transcription was then performed on each of the dilutions.

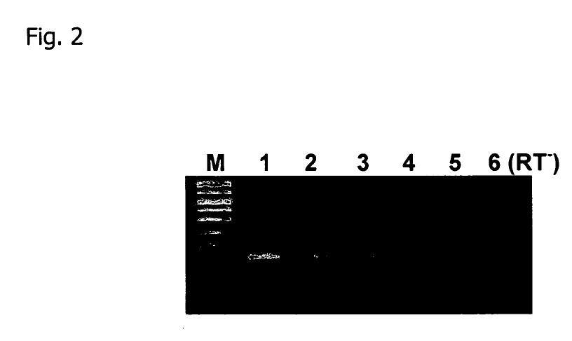

After PCR, the gel electrophoresis image of FIG. 2 was obtained. The lanes correspond to approximately the following amounts of RNA in the reverse transcription reaction:

(1) 1000 ng

(2) 500 ng

(3) 250 ng

(4) 125 ng

(5) 63 ng

(6) 1000 ng RT negative control

### DETAILED DESCRIPTION OF THE INVENTION

The invention is a process for extraction and purification of nucleic acids (RNA and DNA) from a wide variety of samples. The invention uses two possible steps that are based upon competitive binding. In different embodiments of the invention, either or both of these new steps may be incorporated.

In very general terms, the steps of the extraction and purification process are as follows:

1) Cell Lysis

2) Sample Dilution

3) First competitive binding reaction

4) Organic Extraction

5) Nucleic Acid Precipitation and Dissolution

6) Second competitive binding reaction

More specifically, the present invention is present invention is directed to a method of extracting nucleic acids from samples containing prokaryotic cells and/or eukaryotic cells by:

(a) lysing the cells in the sample under conditions that minimize RNA expression or enzymatic degradation of RNA;

(b) dispersing and diluting the lysed cells;

(c) mixing the dispersed and diluted lysed cells with a buffered solution containing cationic and anionic detergents, wherein the anionic detergent facilitates binding of carboxylate containing materials and ionic binding of the nucleic acids is inhibited by the cationic detergent;

(d) separating non-nucleic acid contaminants from the nucleic acids by mixing with an organic extraction solvent to generate an emulsion that separates into an organic phase, an interface containing the contaminants and an aqueous phase containing the nucleic acids;

(e) separating the phases, and retaining the aqueous phase; and

(f) precipitating the nucleic acids in the aqueous phase followed by dissolving the precipitate in nuclease-free solvent.

In an alternative embodiment, the present invention is directed to a method for simultaneously extracting RNA and DNA from samples containing prokaryotic cells and/or eukaryotic cells by:

(a) lysing sample cells;

(b) diluting the lysed cells;

(c) separating cell proteins and contaminant organic matter from the cell nucleic acids by mixing with an organic extraction solvent to generate an emulsion that separates into an organic phase, an interface containing the contaminants and an aqueous phase containing the nucleic acids,

(d) separating the phases formed in step (c) with the aqueous phase being retained;

(e) precipitating the nucleic acids from the aqueous phase followed by dissolving the nucleic acids in a nuclease-free solvent; and

(f) optionally further purifying the nucleic acids by passing the dissolved nucleic acids through a mixture of molecular exclusion resin and anion exchange resin, wherein any remaining organic contaminants are captured while the nucleic acids pass through.

Cell Lysis—Researchers have long recognized that recovery of meaningful mRNA is complicated by the fact that prokaryotic mRNA half-lives are on the order of seconds to minutes. This short half-life necessitates that samples must be frozen in liquid nitrogen immediately upon sampling and maintained in the frozen condition until the nucleic acids are extracted. Traditional extraction methods require sample dissolution prior to cell disruption. This dissolution step allows the samples to warm and consequently permits expression of mRNA, and enzymatic depolymerization of mRNA under laboratory conditions; processes that produce results that do not reflect the actual levels of expressed mRNA in the environment or other sample, at the time of sample collection. For this reason, the sample should remain in a frozen state during lysis to avoid expression. In the preferred embodiment, liquid nitrogen is poured over the sample and physical grinding is used to lyse the cells. A small amount of a solution containing a chaotropic substance such as guanidine can be added in the lysis step to denature proteins and facilitate their separation from nucleic acids. Chaotropic agents tend to increase the solubility of hydrophobic particles in aqueous solutions and these substances are used frequently to destabilize aggregates of non-polar solute particles and micelles, or to denature proteins [Moelbert and De Los Rios, J Chem. Phy., 119(15):7988, (2003)].

Sample dilution—Adsorption of nucleic acids to sample particulates during the extraction process is a significant problem. Dilution of the sample reduces the ratio of particle surface area to extraction volume and therefore reduces the adsorption problem. Again, a chaotropic agent can be added to the dilution buffer to prevent re-folding of proteins, thus keeping the nucleic acids free of proteins and facilitating separation of the protein component of the sample from the nucleic acid component during organic extraction. The ionic strength of the dilution buffer can be adjusted to minimize co-extraction of organic material.

First competitive binding reaction—The diluted sample is prepared for organic extraction by mixing the lysed and dispersed material in a buffered solution containing cationic and anionic detergents. The anionic detergent should be present in slight excess. Competition from the anionic detergent prevents excessive binding of the cationic detergent to the nucleic acids and, therefore, prevents the nucleic acids from collecting at the aqueous-organic interface in the next step.

Organic extraction—A mixture of chloroform and isoamyl alcohol is added to prepare an emulsion. Much of the organic material is bound by the cationic detergent and trapped at the aqueous/organic interface during agitation of the emulsion. The organic and aqueous phases are separated by centrifugation and the aqueous phase containing the nucleic acids is retained.

Nucleic acid precipitation and dissolution—The nucleic acids are precipitated directly from the aqueous phase with an equal volume of isopropanol or other reagent known to those skilled in the art. Isopropanol is preferred over ethanol because a smaller quantity is required and co-precipitation of salts and other unwanted material is avoided. Following centrifugation, the solution is removed and the precipitated nucleic acids are dissolved in a minimal volume of Rnase-free water.

Second competitive binding step—Any remaining organic material in the dissolved nucleic acids can be removed by passing the solution through a resin mixture composed of a molecular exclusion resin and a quaternary amine or other anion exchange moiety known to those skilled in the art. The resin mixture is prepared with a counter-ion that competes with phosphates in the nucleic acids and negative ions in the organic matter for binding to the resin. The competition is based on pH- and salt concentration-dependent differences in stability of ion-exchange complexes. An acetate is a good choice for the counter-ion because, under the correct conditions, it will prevent the nucleic acid phosphate groups from binding to the resin but it will allow carboxylates, the most abundant charged groups in organic matter, to bind. This procedure that uses the resin to bind the impurity instead of the product represents a reversal in the role of anion exchange resin from conventional nucleic acid purification methods.

### EXEMPLIFIED EMBODIMENTS OF THE INVENTION

### Example 1

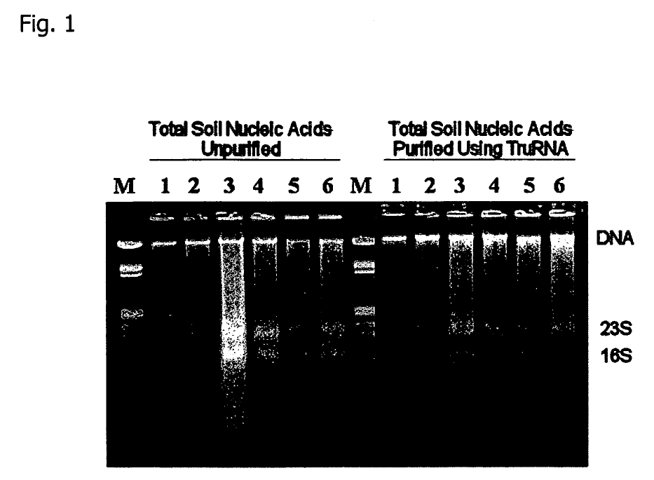

Using the extraction method of the present invention total nucleic acid was extracted from soil samples as follows. FIG. 1 shows the electrophoretic profiles of unpurified total nucleic acids and total nucleic acids following purification with the method of the invention.

Total nucleic acids were extracted from 250 mg samples of soil from two deciduous forest A-horizon soil samples (lanes 1-2), two flood plain sediment samples (lanes 3-4), and two grassy rhizosphere soil samples (lanes 5-6), using the extraction procedure that is the subject of this patent. An aliquot of the total nucleic acid extracts was purified by passage through a 1 cm column prepared with 600 (l of a resin slurry containing Q-Sepharose anion exchange resin and Sephadex G:75 molecular exclusion resin 1:10 that was equilibrated in 50 mM KC2H3O4 pH 5.2 by centrifugation at 500 (g in a spin-filter. The low molecular weight component removed by the subject invention purification method contains fragmented DNA determined RNase/DNase analysis (data not shown).

### Example 2

A single nucleic acid extraction was performed on a deciduous forest A-Horizon soil sample. FIG. 2 shows the agarose gel electrophoresis image of RTPCR product generated from eubacterial glutamine synthetase (glnA) mRNA.

The RNA was separated from the DNA using a QIAGEN RNA/DNA Mini Kit (14123), and separated into two identical aliquots. RNA purification was performed on one fraction by passage through a 1 cm column prepared with 600 (l of a resin slurry containing Q-Sepharose anion exchange resin and Sephadex G:75 molecular exclusion resin 1:10 that was equilibrated in 50 mM KC2H304 pH 5.2. The other RNA fraction was purified using a QIAGEN RNA cleanup procedure supplied with the Mini Kit (14123) that is based on anion exchange resin. The purified RNA fractions were quantified by 260 nm UV absorbance.

5 (g aliquots of the purified RNA samples were treated with 1 U of RQ1 RNase free DNaseI (Promega) for 30 min at 37(C in a volume of 20 (1 in the presence of 2 U (1(1 RNaseOut™ RNase inhibitor (Invitrogen) followed by inactivation at 80 (C for 10 min. Random hexamer primed reverse transcription was performed on 2 (g of each DNase treated RNA sample using 500 U SuperScript II reverse transcriptase in a total reverse transcription reaction volume of 40 (l for 4 h at 42(C. PCR was performed with 2.5 U AmpliTaq Gold DNA polymerase (Invitrogen) in 25 (1 reactions containing 1 (PCR reaction buffer II (Invitrogen) with 2 mM MgCl2, using series two-fold dilutions of the cDNA derived from RNA purified using the method of the subject invention (shown in the figure) and the cDNA derived from RNA purified using the QIAGEN reagent system (not shown) using primers GlnF3 5′aag acc gcg acc ttc atg cc 3′ and GlnR2 5′gat kcc gcc gat gta gta 3′ that generate a 153 or 156 base pair amplicon from a conserved region of the eubacterial glnA gene. PCR conditions used a hot start with an initial dissociation period 4 min at 94 (C followed by 30 cycles consisting of dissociation at 94 (C for 30 s, primer annealing at 58 (C for 1 min and extension at 72 (C for 1 min. Cycling was followed by a final incubation at 72 (C for 10 min. 10 (l of each of the PCR products were electrophoresed at 3V/cm (1 for 2.5 h in 1 (TAE [40 mM Tris(acetate (pH 8.5), 2 mM EDTA] supplemented with 100 ng l (1 ethidium bromide.

After PCR, gel electrophoresis image was obtained and the subject invention method produced the image shown. The commercial cleanup produced a gel image (not shown) with an extremely faint band that was essentially uniform across all lanes. The lanes correspond to approximately the following amounts of RNA in the reverse transcription reaction:

(1) 1000 ng

(2) 500 ng

(3) 250 ng

(4) 125 ng

(5) 63 ng

(6) 1000 ng RT negative control

### REFERENCES CITED

Alm, E. W., D. Zheng, and L. Raskin. The presence of humic substances and DNA in RNA extracts affects hybridization results. Appl. Environ. Microbiol., 66:4547-4554 (2000).
Ausubel, F. M., R. Brent, R. E. Kingston, D. D. Moore, J. G. Seidman, J. A. Smith, and K. Struhl (eds.). 1995. Current Protocols in Molecular Biology. John Wiley & Sons, Inc., New York, N.Y.
Borneman, James and E. W. Triplett. Rapid and direct method for extraction of RNA from soil. Soil Biol. Biochem., 29:1621-1624 (1997).
Chomczynski, P. and N. Sacchi. Single-step method of RNA isolation by acid guanidinium isothiocyanate-phenol-chloroform extraction. Anal. Biochem., 162:156-159 (1987).
Coolen, M. J. L. and J. Overmann. Analysis of subfossil molecular remains of purple sulfur bacteria in lake sediment. Appl. Environ. Microbiol., 64:4513-4521 (1998).
Dell'Anno, A., M. Fabiano, G. C. A. Duineveld, A. Kok, and R. Danovaro. Nucleic acid (DNA, RNA) quantification and RNA/DNA ratio determination in marine sediments: comparison of spectrophotometric, fluorometric, and high-performance liquid chromatography methods and estimation of detrietal DNA. Appl. Environ. Microbiol., 64:3238-3245 (1998).
Duarte, G. F., A. S. Rosado, L Seldin, A. C. Keijzer-Wolters, and J. D. van Elsas. Extraction of riboorganic material RNA and genomic DNA from soil for studying the diversity of the indigenous bacterial community. J. Microbiological Methods, 32:21-29 (1998).
Hayes, M. H. B. 1989. Extraction of humic substances from soil. In Humic Substances in Soil, Sediment, and Water (G. R. Aiken, D. M. McKnight, R. L. Wershaw and P. MacCarthy eds.) John Wiley & Sons NY.
Hurt, R. A., X. Qiu, Y. Roh, A. V. Palumbo, J. M. Teidje, and J. Zhou. Simultaneous extraction of RNA and DNA from environmental samples. Appl. Environ. Microbiol., 67:4495-4503 (2001).
Lee, S.-Y., J. Bollinger, D. Bezdicek, and A. Ogram. Estimation of the abundance of an uncultured soil bacterial strain by a competitive quantitative PCR method. Appl. Environ. Microbiol., 62:3787-3793 (1996).
Leff, L. G., J. R. Dana, J. Vaun McArthur, and L. J. Shimkets. Comparison of methods of DNA extraction from stream sediments. Appl. Environ. Microbiol., 61:1141-1143 (1995).
Lovell, C. R., and Y. Piceno. Purification of DNA from estuarine sediments. J. Microbiol. Methods, 20:161-174 (1994).
Malik, M., J. Kain, C. Pettigrew, and A. Ogram. Purification and molecular analysis of microbial DNA from compost. J. Microbiol. Meth., 20:183-196 (1994).
Moelbert, S. and P. De Los Rios. Chaotropic effect and preferential binding in a hydrophobic interaction model. J Chem. Phy., 119(15):7988-8001 (2003).
Moran, M. A., V. L. Torsvik, T. Torsvik, and R. E. Hodson. Direct extraction and purification of rRNA for ecological studies. Appl. Environ. Microbiol., 59:915-918 (1993).
Ogram, A., G. S. Sayler, and T. Barkay. The extraction and purification of microbial DNA from sediments. J. Microbiol. Methods, 7:57-66 (1987).
Ogram, A., W. Sun, F. J. Brockman, and J. K. Fredrickson. Isolation and characterization of RNA from low-biomass deep-subsurface sediments. Appl. Environ. Microbiol. 61:763-768 (1995).
Purdy, K. J., T. M. Embley, S. Takaii, and D. B. Nedwell. Rapid extraction of DNA and rRNA from sediments by a novel hydroxyapatite spin-column method. Appl. Environ. Microbiol., 62:3905-3907 (1996).
Rondin, M. R., P. R. August, A. D. Betterman, S. F. Brady, T. H. Grossman, M. R. Liles, K. A. Loiacono, B. A. Lynch, I. A. MacNeil, C. Minor, C. L. Tiong, M. Gilman, M. S. Osburne, J. Clardy, J. Handelsman, and R. M. Goodman. Cloning the soil metagenome: a strategy for accessing the genetic and functional diversity of uncultured microorganisms. Appl. Environ. Microbiol., 66:2541-2547 (2000).
Selenska, S. and Walker Klingmüller. Direct recovery and molecular analysis of DNA and RNA from soil. Microb Releases, 1:41-46 (1992).
Zhou, J.-Z., M. A. Bruns, and J. M. Tiedje. DNA recovery from soils of diverse composition. Appl. Environ. Microbiol., 62:316-322 (1996).

We claim:

1. A method of extracting nucleic acids from samples containing prokaryotic cells and/or eukaryotic cells, which comprises:
(a) lysing the cells in the sample under conditions that minimize RNA expression or enzymatic degradation of RNA;
(b) dispersing and diluting the lysed cells;
(c) mixing the dispersed and diluted lysed cells with a buffered solution containing cationic and anionic detergents, wherein the anionic detergent facilitates binding of carboxylate containing materials and ionic binding of the nucleic acids is inhibited by the cationic detergent;
(d) separating non-nucleic acid contaminants from the nucleic acids by mixing with an organic extraction solvent to generate an emulsion that separates into an organic phase, an interface containing the contaminants and an aqueous phase containing the nucleic acids;
(e) separating the phases, and retaining the aqueous phase; and
(f) precipitating the nucleic acids in the aqueous phase followed by dissolving the precipitate in nuclease-free solvent.

2. The method of claim 1 wherein the sample is an environmental sample, clinical specimen, tissue, or cellular culture.

3. The method of claim 1 wherein lysing is performed by physical or chemical means.

4. The method of claim 3 wherein said physical lysing is achieved using a grinding process, ballistic impact, rapid pressure fluctuation, extreme pressure conditions, or milling process.

5. The method of claim 1 where lysing is performed at low temperatures to inhibit protein activity.

6. The method of claim 1, wherein the solution in which the sample is dispersed contains a chaotropic agent and optionally other components that inhibit nuclease activity.

7. The method of claim 1, where said solution comprises 4 M Guanidine thiocyanate, 500 mM 2-mercaptoethanol, 100 mM tris•HCl (pH 7.5) 50 mM EDTA, 1% sarkosyl.

8. The method of claim 1, wherein the anionic and cationic detergents are respectively sarkosyl and cetyltrimethylammonium bromide at weight concentrations of approximately 1% each.

9. The method of claim 1, wherein the organic extraction solvent contains chloroform, or phenol, or a mixture of the two solvents at any proportion, optionally supplemented with 1-5% isoamyl alcohol.

10. The method of claim 1, wherein the solution used to prepare for organic solvent extraction includes a chaotropic agent and optionally other components that inhibit nuclease activity.

11. The method of claim 10, wherein said chaotropic agent and optionally other components that inhibit nuclease activity comprise 6.4 M urea, at least 1 M NaCl, 100 mM tris• (pH 7.0), 50 mM EDTA.

12. A method for simultaneously extracting RNA and DNA from samples containing prokaryotic cells and/or eukaryotic cells, which comprises:
(a) lysing sample cells;
(b) diluting the lysed cells;
(c) separating cell proteins and contaminant organic matter from the cell nucleic acids by mixing with an organic extraction solvent to generate an emulsion that separates into an organic phase, an interface containing the contaminants and an aqueous phase containing the nucleic acids,
(d) separating the phases formed in step (c) with the aqueous phase being retained;
(e) precipitating the nucleic acids from the aqueous phase followed by dissolving the nucleic acids in a nuclease-free solvent; and
(f) optionally further purifying the nucleic acids by passing the dissolved nucleic acids through a mixture of molecular exclusion resin and anion exchange resin, wherein any remaining organic contaminants are captured while the nucleic acids pass through.

13. The method of claim 12, wherein the step (f) is augmented by centrifugal force, gravitational force, or a pressure differential.

14. The method of claim 12, wherein anion exchange resin is a quaternary amino anion-exchange resin that is pre-treated with potassium acetate so that it is in the carboxylate form.

15. The method of claim 12, wherein the resins of step (f) are comprised of an optimized ratio of a molecular exclusion resin and a quaternary amino anion exchange resin.

16. A method for simultaneously extracting RNA and DNA from samples containing prokaryotic cells and/or eukaryotic cells, which comprises:
(a) lysing the cells in the samples;
(b) dispersing and diluting the lysed cells;
(c) preparing for separation by mixing in a buffered solution containing a solution of cationic and anionic detergents
(d) separating cell proteins and organic contaminats by mixing with an organic extraction solvent to generate an emulsion that separates into an organic phase, an interface containing the contaminants and an aqueous phase containing the nucleic acids,
(e) separating the phases, and retaining the aqueous phase;
(f) precipitating the nucleic acids followed by dissolution in a nuclease-free solvent; and
(g) further purifying by passing the dissolved nucleic acids through a mixture of molecular exclusion resin and anion exchange resin, wherein organic contaminants are bind to the resin and the nucleic acids pass through.

17. The method of claim 2, wherein the environmental sample is a soil sample.
